(19) United States
(12) Patent Application Publication
(10) Pub. No.: US 2026/0358498 A1
(43) Pub. Date: Nov. 12, 2026

(54) METHOD AND DEVICE FOR PERFORMING ULTRASONIC IMAGING WITH ROW-COLUMN ADDRESSING

(71) Applicant: MODULEUS, Tours (FR)

(72) Inventors: Pascal CHATAIN, Tours (FR); Audren BOULME, Tours (FR)

(21) Appl. No.: 19/486,824

(22) PCT Filed: May 17, 2024
(86) PCT No.: PCT/EP2024/063641

§ 371 Date: Nov. 21, 2025

(30) Foreign Application Priority Data
May 29, 2023 (FR) FR2305309

(51) Int. Cl. G01S 15/89; G01S 7/524

(52) U.S. Cl. CPC G01S 15/8925; G01S 7/524; G01S 15/8993

(57) ABSTRACT

The present description concerns a method of acquisition of an image of a body by means of a row-column addressing array ultrasonic imaging device (100), the device (100) comprising an array of M rows (Rᵢ) and N columns (Cⱼ) of elementary ultrasonic transducers (101), each row (Rᵢ) being divided into n row portions, each row portion comprising a plurality of transducers.

This application is based on and claims priority of French patent application FR2305309 filed on May 29, 2023, entitled “Procédé et dispositif d'imagerie ultrasonore à adressage ligne-colonne”, which is incorporated herein by reference within the limits provided by law.

### TECHNICAL FIELD

The present disclosure concerns the field of ultrasonic imaging, and more particularly aims at a device comprising a row-column addressing array of ultrasonic transducers, and a method of acquisition of an image of a body by means of such a device.

### PRIOR ART

An ultrasonic imaging device conventionally comprises a plurality of ultrasonic transducers, and an electronic control circuit connected to the transducers. In operation, the assembly of transducers is positioned opposite a body, an image of which is desired to be acquired. The electronic control circuit is configured to apply electrical excitation signals to the transducers, so as to cause the transmission of ultrasonic waves by the transducers, toward the body or object to be analyzed. The ultrasonic waves transmitted by the transducers are reflected by the body to be analyzed (by its inner and/or surface structure), then return to the transducers, which convert them back into electrical signals. These electrical response signals are read by the electronic control circuit and can be stored and analyzed to deduce information about the body being studied.

Ultrasonic transducers can be arranged in a linear array in the case of a two-dimensional image acquisition device, or in a 2D array in the case of a three-dimensional image acquisition device. In the case of a two-dimensional image acquisition device, the acquired image is representative of a cross-section of the body being studied in a plane defined by the alignment axis of the transducers of the linear array on the one hand, and by the transducer transmission direction on the other hand. In the case of a three-dimensional image acquisition device, the acquired image is representative of a volume defined by the two alignment directions of the transducers of the array and by the transducer transmission direction.

Among three-dimensional image acquisition devices, devices known as “fully populated,” in which each transducer in the array is individually addressable can be distinguished from devices known as row-column addressing (RCA) devices, in which the transducers of the array are addressable by row and column.

Fully populated devices offer greater flexibility in the shaping of the ultrasonic beams during transmission and reception. Array control electronics is however complex, the number of transmit/receive channels required being equal to M*N in the case of an array of M rows and N columns. Further, the signal-to-noise ratio is generally relatively low, since each transducer has a small surface area of exposure to ultrasonic waves.

RCA-type devices use different ultrasonic beamforming algorithms. The beamforming possibilities may be decreased as compared with fully populated devices. However, the array control electronics is considerably simplified, the number of required transmit/receive channels being decreased to M+N in the case of an array of M rows and N columns. Further, the signal-to-noise ratio is improved due to the interconnection of the transducers in rows or columns during the transmit and receive phases.

Devices and methods for acquiring three-dimensional images with a row-column addressing (RCA) are here more particularly considered.

International patent application WO2022/268512, previously filed by the applicant, describes a method of acquisition of an image of a body by means of a row-column addressing array ultrasonic imaging device, this method enabling to trace back to the individual contributions of the elementary transducers of the array, so as to benefit both from the advantages of fully populated devices in terms of imaging resolution and from row-column addressing devices in terms of electronic complexity.

It would be desirable to improve, at least partly, certain aspects of the row-column addressing three-dimensional ultrasonic image acquisition devices and methods described in international patent application WO2022/268512 and, more generally, known row-column addressing three-dimensional ultrasonic image acquisition devices and methods.

### SUMMARY OF THE INVENTION

For this purpose, an embodiment provides a method of acquisition of an image of a body by means of a row-column addressing array ultrasonic imaging device, the device comprising an array of M rows and N columns of elementary ultrasonic transducers, each row being divided into n row portions, each row portion a plurality of elementary ultrasonic transducers, with n an integer greater than or equal to two, wherein:
the elementary transducers of a same row portion are connected to a same row portion electrode;
the elementary transducers of distinct row portions are connected to distinct row portion electrodes; and
the elementary transducers of a same column are connected to a same column electrode, the method comprising:
performing p successive firings of a same ultrasonic wave toward the body, where p is an integer designating the number of elementary ultrasonic transducers of the largest row portion;
after each firing, implementing a phase of reception, by means of the device, of a return ultrasonic wave reflected by the body,
wherein, during each of the receive phases, a variable electrical quantity representative of the received wave is read from each row portion electrode of the device, and wherein, between any two receive phases among the p receive phases, the sign of the individual contribution of at least one elementary ultrasonic transducer of the array is changed,
the method further comprising a step of calculating, by means of an electronic processing device, by linear combinations of the variable electrical quantities read from the row portion electrodes of the device during the p receive phases, an individual contribution of each of the elementary transducers of the array.

According to an embodiment, the row portions all comprise the same integer number N/n of elementary ultrasonic transducers.

According to an embodiment, the number M of rows of the array is equal to the number N of columns of the array.

According to an embodiment, the calculation of the individual contributions of the elementary transducers of the array comprises a multiplication of the variable electrical quantities read from the row portion electrodes of the device during the p receive phases by coefficients of an array, determined in advance during a characterization or simulation phase and stored in a memory of the electronic processing device.

According to an embodiment, during each of the p receive phases, each column electrode of the array is maintained at a DC bias voltage, and between any two receive phases among the p receive phases, the sign of the DC bias voltage applied to at least one of the column electrodes of the device is changed.

According to an embodiment, the signs of the bias voltages respectively applied to the column electrodes of the device during the p receive phases are encoded by vectors of an orthogonal matrix, for example a Hadamard matrix.

According to an embodiment, the p successive firings are performed by means of the row-column addressing array ultrasonic imaging device, and, for each of the p firings, each column electrode of the array is maintained at a DC bias voltage, and an AC excitation voltage superimposed on the DC bias voltage is applied to said column electrode, and, for each firing, the signs of the DC bias voltages respectively applied to the column electrodes of the array are the same as the signs of the DC bias voltages respectively applied to the column electrodes during the subsequent receive phase.

According to an embodiment, for each firing, a same transmit circuit simultaneously applies a same DC bias voltage and a same AC excitation voltage to the column electrodes of n columns of the array.

According to an embodiment, the ultrasonic transducers are CMUT or PMUT transducers.

According to an embodiment, n is equal to two.

According to an embodiment, the variable electrical quantity read from each row portion electrode of the device is a voltage.

Another embodiment provides a row-column addressing array ultrasonic imaging device, the device comprising an array of M rows and N columns of elementary ultrasonic transducers, each row being divided into n row portions, each row portion comprising a plurality of elementary ultrasonic transducers, with n an integer greater than or equal to two, wherein:
the elementary transducers of a same row portion are connected to a same row portion electrode;
the elementary transducers of distinct row portions are connected to distinct row portion electrodes; and
the elementary transducers of a same column are connected to a same column electrode,
   the device further comprising a control circuit configured to implement a method such as defined hereabove.

According to an embodiment, the device comprises M*n receive circuits respectively connected to the M*n row portion electrodes of the array of elementary ultrasonic transducers.

According to an embodiment, the device comprises an integer number s smaller than N of transmit circuits, where a same transmit circuit can be simultaneously connected to elementary transducer column electrodes belonging to distinct row portions of a same row of the array.

According to an embodiment, the row portions all comprise the same integer number N/n of elementary ultrasonic transducers, and the number s of transmit circuits is equal to N/n.

### BRIEF DESCRIPTION OF THE DRAWINGS

The foregoing features and advantages, as well as others, will be described in detail in the rest of the disclosure of specific embodiments given as an illustration and not limitation with reference to the accompanying drawings, in which:

FIG. 1 is a top view schematically and partially showing an example of a row-column addressing array ultrasonic imaging device;

FIG. 2 comprises a cross-section view along a plane A-A of FIG. 1 and a cross-section view along a plane B-B of FIG. 1, illustrating in more detail an example of embodiment of the device of FIG. 1;

FIG. 3 is an equivalent electrical diagram of a 2×2 row-column addressing array ultrasonic imaging device operating in receive mode;

FIG. 4 is an equivalent electrical diagram of an N×N row-column addressing array ultrasonic imaging device operating in receive mode;

FIG. 5 schematically illustrates a transmit step TX and a receive step RX of an implementation mode of a method of acquiring an image of a body by means of an N×N row-column addressing array ultrasonic imaging device according to an embodiment;

FIG. 6 schematically illustrates steps of a method of acquisition of an image of a body by means of a 2×2 row-column addressing array ultrasonic imaging device according to an embodiment;

FIG. 7 schematically illustrates steps of a method of acquisition of an image of a body by means of a 4×4 row-column addressing array ultrasonic imaging device according to an embodiment; and

FIG. 8 is a cross-section view of an example of a row-column addressing array ultrasonic imaging device according to an embodiment.

### DESCRIPTION OF EMBODIMENTS

The same elements have been designated by the same references in the various figures. In particular, structural and/or functional elements common to the different embodiments may have the same references and may have identical structural, dimensional and material properties.

For the sake of clarity, only those steps and elements that are useful for understanding the described embodiments have been shown and have been described in detail. In particular, the various possible applications of the described imaging devices and methods have not been detailed, the described embodiments being compatible with usual applications of ultrasonic imaging solutions. In particular, the properties (frequencies, shapes, amplitudes, etc.) of the electrical excitation signals applied to the ultrasonic transducers have not been detailed, the described embodiments being compatible with excitation signals commonly used in ultrasonic imaging systems, which can be selected according to the considered application and, in particular, to the nature of the body to be analyzed and the type of information which is desired to be acquired. Similarly, the various processes applied to the electrical signals delivered by the ultrasonic transducers to extract useful information about the body to be analyzed have not been detailed, the described embodiments being compatible with the processes currently implemented in ultrasonic imaging systems. Further, the circuits for controlling the ultrasonic transducers of the described imaging devices have not been detailed, the embodiments being compatible with all or most known circuits for controlling ultrasonic transducers of row-column addressing array ultrasonic imaging devices, or the forming of these circuits being within the abilities of those skilled in the art on reading of the present disclosure. Further, the forming of the ultrasonic transducers of the described imaging devices has not been detailed, the described embodiments being compatible with all or most known ultrasonic transducer structures.

Unless otherwise specified, when reference is made to two elements being connected to each other, this means directly connected without any intermediate elements other than conductors, and when reference is made to two elements being coupled to each other, this means that these two elements may be connected or may be connected via one or more other elements.

In the following description, where reference is made to absolute position qualifiers, such as the terms “front”, “back”, “top”, “bottom”, “left”, “right”, etc., or relative position qualifiers, such as the terms “top”, “bottom”, “upper”, “lower”, etc., or orientation qualifiers, such as “horizontal”, “vertical”, etc., reference is made unless otherwise specified to the orientation of the drawings.

Unless specified otherwise, the expressions “about”, “approximately”, “substantially”, and “in the order of” signify plus or minus 10%, preferably of plus or minus 5%.

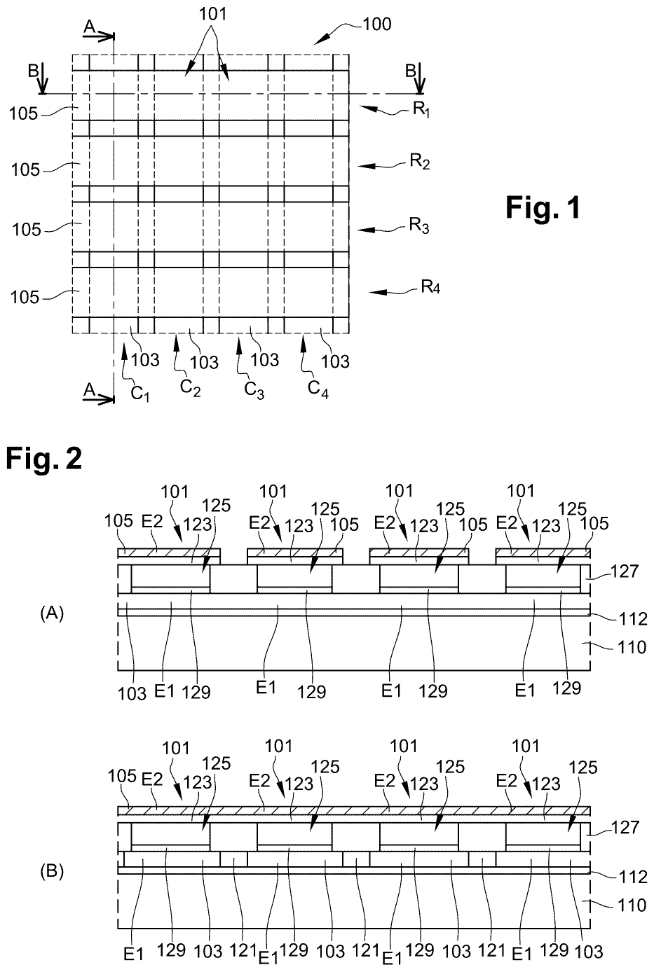

FIG. 1 is a top view schematically and partially showing an example of a row-column addressing array ultrasonic imaging device 100.

FIG. 2 comprises two cross-section views (A) and (B) of the device 100 of FIG. 1 respectively along planes A-A and B-B of FIG. 1.

Device 100 comprises a plurality of ultrasonic transducers 101 arranged in an array of M rows Rᵢ and N columns Cⱼ, with M and N integers greater than or equal to 2, i an integer ranging from 1 to M, and j an integer ranging from 1 to N.

In FIG. 1, four rows R₁, R₂, R₃, R₄ and four columns C₁, C₂, C₃, C₄ are shown. In practice, the numbers M of rows and N of columns of device 100 may be different from 4.

Each transducer 101 of device 100 comprises a lower electrode E1 and an upper electrode E2 (FIGS. 2A and 2B). When an appropriate excitation voltage is applied between its electrodes E1 and E2, the transducer transmits an ultrasonic acoustic wave. When the transducer receives an ultrasonic acoustic wave within a given frequency range, it delivers between its electrodes E1 and E2 a voltage representative of the received wave.

In this example, transducers 101 are capacitive membrane transducers, also known as CMUT transducers (Capacitive Micromachined Ultrasonic Transducers).

In each column Cⱼ of the transducer array, the transducers 101 in the column have their respective lower electrodes E1 connected to each other. The lower electrodes E1 of transducers 101 of separate columns are however not connected to each other. Further, in each row Rᵢ of the transducer array, the transducers 101 in the row have their respective upper electrodes E2 connected to one another. The upper electrodes E2 of transducers 101 of separate rows are however not connected to one another.

In each column Cⱼ of device 100, the lower electrodes E1 of the transducers 101 in the column form a continuous conductive or semiconductor strip 103, extending along substantially the entire length of the column. As a variant, each strip 103 of electrodes E1 comprises a vertical stack of a semiconductor strip and of a conductive strip, each extending substantially along the entire length of the column. Further, in each row Rᵢ of device 100, the upper electrodes E2 of the transducers 101 in the row form a continuous conductive or semiconductor strip 105 extending along substantially the entire length of the row. As a variant, each strip 105 of electrodes E2 comprises a vertical stack of a semiconductor strip and of a conductive strip, each extending substantially along the entire length of the row. For the sake of simplicity, only the lower electrode strips 103 and upper electrode strips 105 are shown in FIG. 1.

In the shown example, the strips 103 forming the column electrodes are made of a doped semiconductor material, for example of doped silicon. Further, in this example, the strips 105 forming the row electrodes are made of metal. For example, in top view, the lower strips 103 are parallel to one another, and the upper strips 105 are parallel to one another and perpendicular to strips 103.

In the example of FIG. 1, device 100 comprises a support substrate 110, for example made of a semiconductor material, for example silicon. The array of ultrasonic transducers 101 is arranged on the upper surface of substrate 110. More particularly, in this example, a dielectric layer 112, for example a silicon oxide layer, forms an interface between substrate 110 and the array of ultrasonic transducers 101. Dielectric layer 112 extends, for example, continuously, over the entire upper surface of support substrate 110. As an example, layer 112 is in contact, by its lower surface, with the upper surface of substrate 110, over substantially the entire upper surface of substrate 110.

The lower electrode strips 103 are arranged on the upper surface of dielectric layer 112, for example in contact with the upper surface of dielectric layer 112. Strips 103 may be laterally separated from one another by dielectric strips 121, for example made of silicon oxide, extending parallel to strips 103 and having a thickness substantially identical to that of strips 103.

Each transducer 101 comprises a cavity 125 formed in a rigid support layer 127, and a flexible membrane 123 suspended above cavity 125. Layer 127 is, for example, a silicon oxide layer. Layer 127 is arranged on the upper surface, for example substantially planar, of the assembly formed by alternating strips 103 and 121. In each transducer 101, cavity 125 is located opposite the lower electrode E1 of the transducer.

In the shown example, each transducer 101 comprises a single cavity 125 opposite its lower electrode E1. As a variant, in each transducer 101, cavity 125 may be divided into a plurality of elementary cavities, for example arranged, in top view, in an array of rows and columns, laterally separated from one another by side walls formed by portions of layer 127.

In the shown example, at the bottom of each cavity 125, a dielectric layer 129, for example made of silicon oxide, coats the lower electrode E1 of the transducer, so as to prevent any electrical contact between flexible membrane 123 and the lower electrode E1 of the transducer. As a variant, to ensure this electrical insulation function, a dielectric layer (not shown) may coat the lower surface of membrane 123. In this case, layer 129 may be omitted.

In each transducer 101, flexible membrane 123, coating the transducer cavity 125, is, for example, made of a doped or undoped semiconductor material, for example, of silicon.

In each transducer 101, the upper electrode E2 of the transducer is arranged on top of and in contact with the upper surface of the flexible membrane 123 of the transducer, vertically in line with cavity 125 and the lower electrode E1 of the transducer. As a variant, in the case of a semiconductor membrane, the upper electrode E2 of each transducer 101 may be formed by the membrane itself, in which case layer 105 may be omitted.

As an example, in each row Rᵢ of device 100, the flexible membranes 123 of the transducers 101 in the row form a continuous membrane strip extending along substantially the entire length of the row, laterally separated from the membrane strips of neighboring rows by a dielectric region. In each row Rᵢ, the membrane strip 123 of the row coincides, for example, in top view, with the upper electrode strip 105 of the row.

For each row Rᵢ of transducer array 101, device 100 may comprise a transmit circuit, a receive circuit, and a controllable switch for, in a first configuration, connecting the electrodes E2 of the transducers in the row to an output terminal of the transmit circuit of the row, and, in a second configuration, connecting the electrodes E2 of the transducers in the row to an input terminal of the receive circuit of the row.

Further, for each column Cⱼ of the array of transducers 101, device 100 may comprise a transmit circuit, a receive circuit, and a controllable switch for, in a first configuration, connecting the electrodes E1 of the transducers in the column to an output terminal of the transmit circuit of the column, and, in a second configuration, connecting the electrodes E1 of the transducers in the column to an input terminal of the receive circuit of the column.

For simplicity, the transmit and receive circuits and the switches of device 100 have not been shown in the drawings. Further, the implementation of these elements has not been detailed, the described embodiments being compatible with usual implementations of transmit/receive circuits of row-column addressing array ultrasonic imaging devices. As a non-limiting example, the transmit/receive circuits may be identical or similar to those described in French patent application No. 19/06515 filed by the applicant on Jun. 18, 2019.

The acquisition of an ultrasonic image of a body by means of a row-column addressing array ultrasonic imaging device, for example of the type described in relation with FIGS. 1 and 2, may comprise a phase of transmission of an ultrasonic wave, followed by a phase of reception of a return ultrasonic wave reflected by the body.

As an example, during the transmit phase, the row electrodes of the transducer array (corresponding to electrodes 105 in the example of FIGS. 1 and 2) are maintained at a fixed reference potential, for example ground, and a DC bias potential Vbias is applied to each of the column electrodes of the array (corresponding to electrodes 103 in the example of FIGS. 1 and 2). An AC excitation voltage V_{exc} is also applied to each of the column electrodes of the array. Thus, each transducer 101 of the array sees, between its electrodes E1 and E2, AC excitation voltage V_{exc} Superimposed to a DC bias voltage V_{bias}. This causes a vibration of the transducer membrane, leading to the emission of an ultrasonic acoustic wave.

During the receive phase, the column electrodes may be maintained at DC bias potential V_{bias}. An AC voltage superimposed on DC bias voltage V_{bias} then appears between the electrodes E1 and E2 of each transducer 101 under the effect of the return acoustic wave. The AC voltages generated individually by the elementary transducers of the array, also called individual contributions of the elementary transducers, combine on the row and column electrodes of the array, and the resulting voltages can be read from said row and column electrodes.

The above-mentioned international patent application WO2022/268512, the content of which is incorporated herein by reference, describes a method of acquisition of an ultrasonic image by means of a row-column addressing array ultrasonic imaging device, enabling to trace back to the individual contributions of the elementary transducers of the array, so as to benefit both from the advantages of fully populated devices in terms of imaging resolution and of row-column addressing devices in terms of electronic complexity.

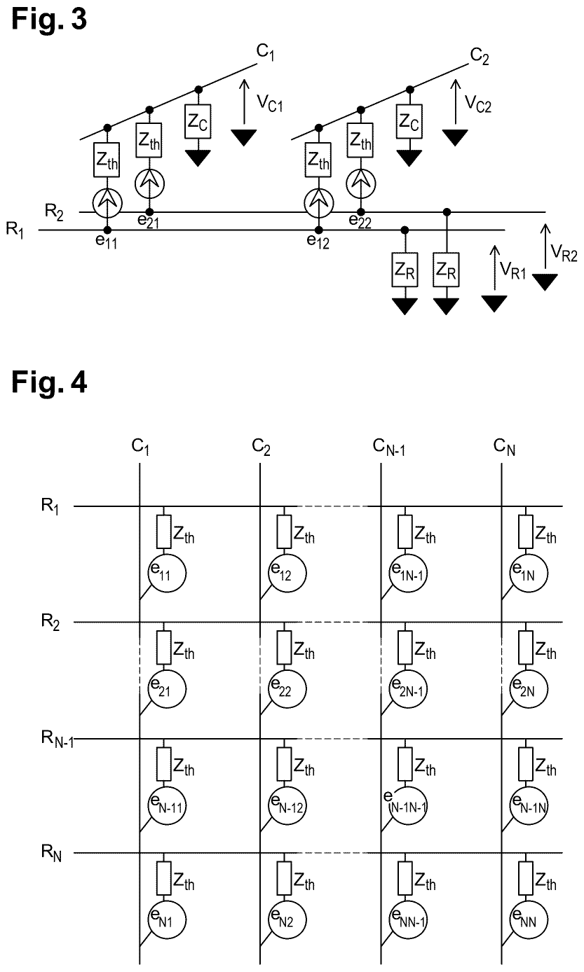

FIG. 3 is an equivalent electrical diagram of a 2-row, 2-column row-column addressing array ultrasonic imaging device during an ultrasonic wave reception phase.

Each elementary transducer may be modeled by a voltage generator eᵢⱼ in series with an impedance Zₜₕ between a row electrode Rᵢ (corresponding to an electrode 105 in the example of implementation of FIGS. 1 and 2) and a column electrode Cⱼ (corresponding to electrode 103 in the example of implementation of FIGS. 1 and 2). Each voltage value eᵢⱼ is representative of the vibration of the elementary transducer of coordinates i,j in the array under the effect of the received ultrasonic wave, and corresponds to the individual contribution of the elementary transducer to the voltages measured on the row and/or column electrodes of the device. There has further been shown, for each row of the array, a load impedance Z_{R} between row electrode Rᵢ and ground, and for each column of the array, a load impedance Z_{C} between column electrode Cⱼ and ground. The impedances Zₜₕ of the various elementary transducers of the array are, for example, all identical or substantially identical. The load impedances Z_{R} connected to the various row electrodes Rᵢ of the array are, for example, all identical or substantially identical. The load impedances Z_{C} connected to the various column electrodes Cⱼ of the array are, for example, all identical or substantially identical. There is here designated by V_{Ri} the AC voltage appearing on row Rᵢ during an ultrasonic wave reception phase, and by V_{Cj} the AC voltage appearing on column Cⱼ during an ultrasonic wave reception phase.

By application of the superposition principle, the following equation system can be written:

$$\{ \begin{matrix}{V}_{R 1} = {a}_{11 R 1} * {e}_{11} + {a}_{12 R 1} * {e}_{12} + {a}_{21 R 1} * {e}_{21} + {a}_{22 R 1} * {e}_{22} \\ {V}_{R 2} = {a}_{11 R 2} * {e}_{11} + {a}_{12 R 2} * {e}_{12} + {a}_{21 R 2} * {e}_{21} + {a}_{22 R 2} * {e}_{22} \\ {V}_{C 1} = {a}_{11 C 1} * {e}_{11} + {a}_{12 C 1} * {e}_{12} + {a}_{21 C 1} * {e}_{21} + {a}_{22 C 1} * {e}_{22} \\ {V}_{C 2} = {a}_{11 C 2} * {e}_{11} + {a}_{12 C 2} * {e}_{12} + {a}_{21 C 2} * {e}_{21} + {a}_{22 C 2} * {e}_{22}\end{matrix} \qquad [ Math 1 ]$$

where each coefficient a_{ijRk} is representative of the weight of the contribution of generator eᵢⱼ to the voltage V_{Rk} of row Rₖ, with k an integer ranging from 1 to N, and where each coefficient a_{ijck} is representative of the weight of the contribution of generator eᵢⱼ to the voltage V_{Ck} of column Cₖ.

FIG. 4 is an equivalent electrical diagram of an N-row, N-column row-column addressing array ultrasonic imaging device. The equivalent electrical diagram of FIG. 4 is similar to that of FIG. 2. For simplification, the load impedances Z_{R} connected to the rows and the load impedances Z_{C} connected to the columns have not been shown in FIG. 4.

Using the previous notations, the above equation system [Math 1] can be rewritten in array form as follows:

[A]*[e]=[V]

With:

$$[ A ] = [ \begin{matrix}{a}_{11 R 1} & {a}_{12 R 1} & … & {a}_{N ( N - 1 ) R 1} & {a}_{NNR 1} \\ {a}_{11 R 2} & {a}_{12 R 2} & … & {a}_{N ( N - 1 ) R 2} & {a}_{NNR 2} \\ … & … & … & … & … \\ {a}_{11 R N} & {a}_{12 R N} & … & {a}_{N ( N - 1 ) R N} & {a}_{N N R N} \\ {a}_{11 C 1} & {a}_{12 C 1} & … & {a}_{N ( N - 1 ) C 1} & {a}_{NNC 1} \\ … & … & … & … & … \\ {a}_{11 C ( N - 1 )} & {a}_{12 C ( N - 1 )} & … & {a}_{N ( N - 1 ) C ( N - 1 )} & {a}_{N N C ( N - 1 )} \\ {a}_{11 C N} & {a}_{12 C N} & … & {a}_{N N - 1 CN} & {a}_{N N C N}\end{matrix} ] \qquad [ Math 2 ]$$

And:

$$[ e ] = [ \begin{matrix}{e}_{11} \\ {e}_{12} \\ ⋮ \\ {e}_{NN - 1} \\ {e}_{NN}\end{matrix} ] \qquad [ Math 3 ]$$

And:

$$[ V ] = [ \begin{matrix}{V}_{R 1} \\ ⋮ \\ {V}_{RN} \\ {V}_{C 1} \\ ⋮ \\ {V}_{CN}\end{matrix} ] \qquad [ Math 4 ]$$

The 2N*(N*N) coefficients of array [A] can be determined in advance during a device characterization or simulation phase and stored in the memory of an electronic processing device. The 2N*(N*N) coefficients of array [A] are, for example, non-zero and non-unit (in absolute value).

The 2N voltages of vector [V] can be read from the row and column electrodes of the array.

A system with 2N equations and N*N unknowns (the N*N voltage values eᵢⱼ) is thus obtained.

As it stands, this system cannot be solved, the number of independent equations being smaller than the number of unknowns.

In above-mentioned international patent application WO2022/268512, it is provided to increase the number of discriminating equations in the system by performing a plurality of successive firings of the same ultrasonic wave (that is, a beam with the same characteristics) towards the body to be analyzed, and by each time changing, during the phase of reception of the return wave, the sign of at least one generator eᵢⱼ.

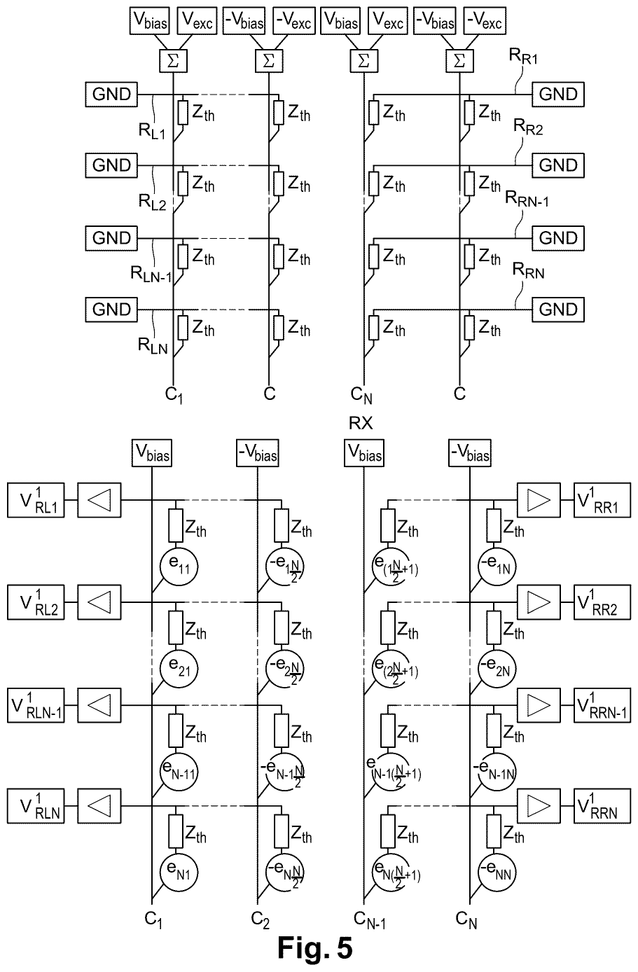

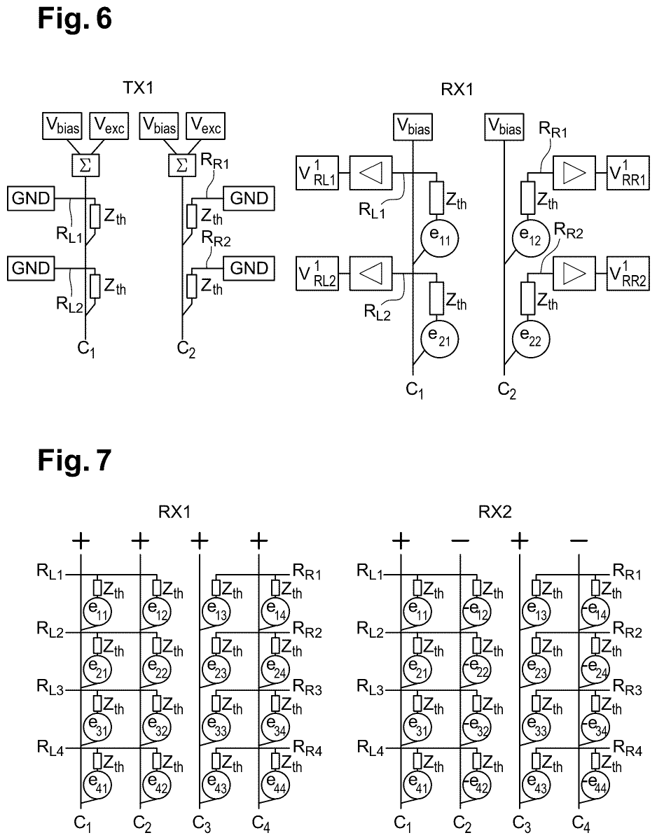

More particularly, it is provided, at each firing, to change the sign of the bias voltage applied to at least one of the column electrodes of the array during the phase of reception of the return wave, so as to reverse the sign of all the generators eᵢⱼ of said at least one column. Advantage is thus taken of the symmetrical voltage behavior of CMUT transducers, explained in relation with FIGS. 5 and 6 of international patent application WO2022/268512.

By performing a plurality of successive firings of the same ultrasonic wave, each time changing the sign of the bias voltages applied to certain columns in receive phase, and by performing linear combinations of the voltages measured on row electrodes Rᵢ, it is possible to trace back to the individual contributions eᵢⱼ of each of the elementary transducers of the array.

A limitation of the solution proposed in above-mentioned international patent application WO2022/268512 lies in the relatively high number of successive firings of the same ultrasonic wave to be implemented to trace back to the individual contributions eᵢⱼ of each of the elementary transducers of the array.

More particularly, for an array of N rows and N columns of elementary ultrasonic transducers, the solution provided in above-mentioned international patent application WO2022/268512 requires implementing at least N successive firings of a same ultrasonic wave, each time changing the sign of the bias voltage applied to at least one of the column electrodes of the array during the phase of reception of the return wave, to trace back to the individual contributions eᵢⱼ of each of the N*N elementary transducers of the array.

FIG. 5 schematically illustrates a transmit step TX and a receive step RX of a method of acquisition of an image of a body by means of an N×N row-column addressing array ultrasonic imaging device, according to an embodiment.

The acquisition device of FIG. 5 differs from the devices previously described in relation with FIGS. 1 to 4 in that, in the example of FIG. 5, each row Rᵢ is divided into two row portions R_{Li} and R_{Ri}, respectively corresponding to a left-hand portion and a right-hand portion of the row. Row portions R_{Li} and R_{Ri} each comprise, for example, N/2 elementary transducers. In this example, N is an even integer.

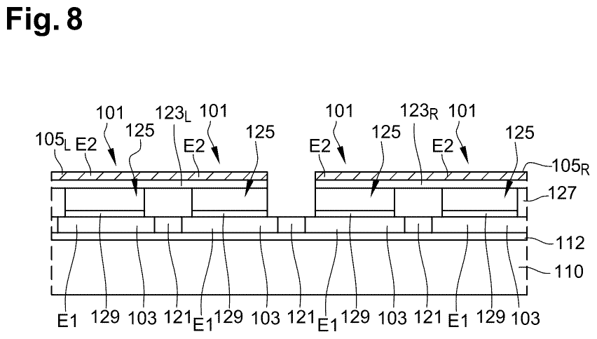

FIG. 8 is a simplified and partial cross-section view illustrating in more detail an example of embodiment of the row-column addressing array ultrasonic imaging device of FIG. 5. FIG. 8 is a cross-section view of the device in the same plane as view (B) of FIG. 2.

As in the examples previously described in relation with FIGS. 1 and 2, in each column Cⱼ of the transducer array, the transducers 101 in the column have their respective lower electrodes E1 connected to each other. The lower electrodes E1 of transducers 101 of separate columns are however not connected to one another.

Further, in each row portion R_{Li}, respectively R_{Ri}, Of the transducer array, the transducers 101 of the considered row portion have their respective upper electrodes E2 connected to one another. The upper electrodes E2 of transducers 101 of separate row portions are however not connected to one another.

As in the example previously described in relation with FIGS. 1 and 2, in each column Cⱼ of the device, the lower electrodes E1 of the transducers 101 in the column form a continuous conductive and/or semiconductor strip 103, extending substantially along the entire length of the column.

Further, in each row portion R_{Li}, respectively R_{Ri}, Of the device, the upper electrodes E2 of the transducers 101 of the row portion form a continuous conductive and/or semiconductor strip 105L, respectively 105R, extending substantially along the entire length of the row portion.

As an example, the device of FIG. 8 differs from the device 100 described in relation with FIGS. 1 and 2 essentially in that, in the device of FIG. 8, in each row of the ultrasonic transducer array, upper electrode strip 105 is interrupted substantially at its center, and thus divided into two separate strips 105L and 105R (not electrically connected to each other).

Similarly, in the case where the membranes 123 of the transducers of a same row form a continuous strip of conductive or semiconductor material, in each row of the ultrasonic transducer array, membrane strip 123 may be interrupted substantially at its center, and thus divided into two separate strips 123L and 123R. The membrane strips 123L and 123R of each row coincide, for example, respectively, in top view, with the upper electrode strips 105L and 105R of the row.

As an example, for each row portion R_{Li}, respectively R_{Ri}, of transducer array 101, the device may comprise a receive circuit having an input terminal connected or coupled, for example via a switch, to the electrodes E2 of the transducers of the row portion.

Further, for each column Cⱼ of transducer array 101, the device may comprise a transmit circuit, for example a pulse generator, having an output terminal connected or coupled, for example via a switch, to the electrodes E1 of the transducers in the column.

Thus, in this as example, compared with a configuration of the type described in relation with FIGS. 1 to 4, the number of row receive circuits is doubled (two receive circuits per row of the array instead of a single one in the examples of FIGS. 1 to 4). The number of column transmit circuits remains unchanged (one transmit circuit per column of the array).

For simplification, the transmit and receive circuits have not been shown in the drawings.

In the example of FIG. 5, during transmit phase TX, a DC bias voltage V_{bias} and an AC excitation voltage V_{exc} are applied to the column electrodes C of odd rank, and an opposite DC bias voltage-V_{bias} and an opposite AC excitation voltage-V_{exc} are applied to the column electrodes C of even rank. For example, during the transmit phase, the electrodes of the row portions R_{Li}, RR of the transducer array (corresponding to electrodes 105L, 105R in the example of FIG. 8) are maintained at a fixed reference potential, for example ground. In FIG. 5, each elementary transducer is represented by an impedance Zₜₕ during transmit phase TX.

During receive phase RX, the DC bias voltages V_{bias} and −V_{bias} respectively applied to the column electrodes Cⱼ of odd rank and to the column electrodes Cⱼ of even rank remain unchanged.

As explained in relation with FIGS. 5 and 6 of the above-mentioned patent application WO2022/268512, the reversal of the sign of voltages V_{bias} and V_{exc} on the columns of even rank has no impact on the acoustic wave emitted by the transducers in the column. In other words, the emitted ultrasonic wave is the same as if the signs of voltages V_{bias} and V_{exc} were the same on all columns.

In receive mode, the reversal of the sign of the DC bias voltage in columns of even rank however leads to reversing the signs of the contributions eᵢⱼ of the elementary transducers of said columns.

By performing a plurality of successive firings of a same ultrasonic wave, each time changing the sign of the bias voltages applied to certain columns in receive phase, and by performing linear combinations of the voltages measured on row electrodes R_{Li}, R_{Ri}, it is possible to trace back to the individual contributions eᵢⱼ of each of the elementary transducers of the array.

The division of each row of the array into two row portions R_{Li}, R_{Ri} advantageously enables to divide by two the number of firings required to trace back to the individual contributions eᵢⱼ of each of the elementary transducers in the array.

Thus, in the example of FIG. 5, N/2 successive firings of a same ultrasonic wave enable to trace back to the individual contributions eᵢⱼ of all the elementary transducers of the array.

More generally, the described embodiments enable to trace back to the individual contributions eᵢⱼ of each of the elementary transducers of the array with a number of firings smaller than N.

FIG. 6 schematically illustrates an example of an implementation mode of a method of acquisition of an image of a body by means of a 2×2 row-column addressing array ultrasonic imaging device of the type described in relation with FIG. 5.

The method of FIG. 6 comprises a single firing of an ultrasonic wave toward the body to be analyzed.

More specifically, the method comprises a first step of transmission TX1 of the ultrasonic wave, followed by a first step of reception RX1 of a return wave reflected by the body to be analyzed.

During transmit step TX1, the column electrodes C₁ and C₂ are biased at the same DC voltage V_{bias} and receive the same AC excitation voltage V_{exc} superimposed on voltage V_{bias}.

During receive phase RX1, column electrodes C₁ and C₂ remain biased to voltage V_{bias}, and AC voltages V_{RL1}¹ and V_{RR1}¹ are respectively read from the electrodes of row portions R_{L1} and R_{R1}, and AC voltages V_{RL2}¹ and V_{RR2}¹ are respectively read from the electrodes of row portions R_{L2} and R_{R2}.

The following equation systems can thus be written:

$$\{ \begin{matrix}{V}_{RL 1}^{1} = {a}_{11 RL 1} * {e}_{11} + {a}_{21 RL 1} * {e}_{21} \\ {V}_{RL 2}^{1} = {a}_{11 RL 2} * {e}_{11} + {a}_{21 RL 2} * {e}_{21}\end{matrix} \qquad [ Math 5 ]$$

$$\{ \begin{matrix}{V}_{RR 1}^{1} = {a}_{12 RR 1} * {e}_{12} + {a}_{22 RR 1} * {e}_{22} \\ {V}_{R R 2}^{1} = {a}_{12 RR 2} * {e}_{12} + {a}_{22 RR 2} * {e}_{22}\end{matrix} \qquad [ Math 6 ]$$

A system of four equations and four unknowns that can be directly solved is thus obtained, which enables to determine the respective contributions e₁₁, e₁₂, e₂₁, and e₂₂ of the elementary transducers.

FIG. 7 schematically illustrates, as an illustrative example, an implementation mode of a method of acquisition of an image of a body by means of a 4×4 row-column addressing array ultrasonic imaging device of the type described in relation with FIG. 5.

FIG. 7 more particularly shows the signals of the bias voltages applied to the column electrodes C of the array during two successive receive phases RX1 and RX2 of the method. In this drawing, a + sign has been shown at the top of the column when the bias voltage applied to the column is equal to V_{bias}, and a − sign has been shown at the top of the column when the bias voltage applied to the column is equal to V_{bias}.

During the receive phase RX1 following the first firing, column electrodes C₁, C₂, C₃, and C₄ are all positively biased (++++). During the receive phase RX2 following the second firing, column electrodes C₁, C₂, C₃, and CA are respectively positively, negatively, positively and negatively biased (+−+−).

For each of receive phases RX1 and RX2, two equation systems of the same type as the above-described systems [Math 5] and [Math 6] can be written, with, each time, 4 equations per system and 8 terms d_{ijRLi}*eᵢⱼ Or d_{ijRRi}*eᵢⱼ per equation.

For each ultrasonic firing, a set of 8 equations and 16 unknowns is thus obtained.

With two ultrasonic firings, 16 equations for 16 unknowns are thus obtained, which enables to trace back to the individual contributions eᵢⱼ of all the elementary transducers.

More particularly, in the shown example, for receive phase RX1, one obtains the following systems:

$$\{ \begin{matrix}\begin{matrix}{V}_{RL 1}^{1} = {a}_{11 RL 1} * {e}_{11} + {a}_{12 RL 1} * {e}_{12} + {a}_{21 RL 1} * {e}_{21} + {a}_{22 RL 1} * \\ {e}_{22} + {a}_{31 RL 1} * {e}_{31} + {a}_{32 RL 1} * {e}_{32} + {a}_{41 RL 1} * {e}_{41} + {a}_{42 RL 1} * {e}_{42}\end{matrix} \\ \begin{matrix}{V}_{R L 2}^{1} = {a}_{11 RL 2} * {e}_{11} + {a}_{12 RL 2} * {e}_{12} + {a}_{21 RL 2} * {e}_{21} + {a}_{22 RL 2} * \\ {e}_{22} + {a}_{31 RL 2} * {e}_{31} + {a}_{32 RL 2} * {e}_{32} + {a}_{41 RL 2} * {e}_{41} + {a}_{42 RL 2} * {e}_{42}\end{matrix} \\ \begin{matrix}{V}_{R L 3}^{1} = {a}_{11 RL 3} * {e}_{11} + {a}_{12 RL 3} * {e}_{12} + {a}_{21 RL 3} * {e}_{21} + {a}_{22 RL 3} * \\ {e}_{22} + {a}_{31 RL 3} * {e}_{31} + {a}_{32 RL 3} * {e}_{32} + {a}_{41 RL 3} * {e}_{41} + {a}_{42 RL 3} * {e}_{42}\end{matrix} \\ \begin{matrix}{V}_{R L 4}^{1} = {a}_{11 RL 4} * {e}_{11} + {a}_{12 RL 4} * {e}_{12} + {a}_{21 RL 4} * {e}_{21} + {a}_{22 RL 4} * \\ {e}_{22} + {a}_{31 RL 4} * {e}_{31} + {a}_{32 RL 4} * {e}_{32} + {a}_{41 RL 4} * {e}_{41} + {a}_{42 RL 4} * {e}_{42}\end{matrix}\end{matrix} \qquad [ Math 5 ]$$

$$\{ \begin{matrix}\begin{matrix}{V}_{RR 1}^{1} = {a}_{13 RR 1} * {e}_{13} + {a}_{14 RR 1} * {e}_{14} + {a}_{23 RR 1} * {e}_{23} + {a}_{24 RR 1} * \\ {e}_{24} + {a}_{33 RR 1} * {e}_{33} + {a}_{34 RR 1} * {e}_{34} + {a}_{43 RR 1} * {e}_{43} + {a}_{44 RR 1} * {e}_{44}\end{matrix} \\ \begin{matrix}{V}_{RR 2}^{1} = {a}_{13 RR 2} * {e}_{13} + {a}_{14 RR 2} * {e}_{14} + {a}_{23 RR 2} * {e}_{23} + {a}_{24 RR 2} * \\ {e}_{24} + {a}_{33 RR 2} * {e}_{33} + {a}_{34 RR 2} * {e}_{34} + {a}_{43 RR 2} * {e}_{43} + {a}_{44 RR 2} * {e}_{44}\end{matrix} \\ \begin{matrix}{V}_{RR 3}^{1} = {a}_{13 RR 3} * {e}_{13} + {a}_{14 RR 3} * {e}_{14} + {a}_{23 RR 3} * {e}_{23} + {a}_{24 RR 3} * \\ {e}_{24} + {a}_{33 RR 3} * {e}_{33} + {a}_{34 RR 3} * {e}_{34} + {a}_{43 RR 3} * {e}_{43} + {a}_{44 RR 3} * {e}_{44}\end{matrix} \\ \begin{matrix}{V}_{RR 4}^{1} = {a}_{13 RR 4} * {e}_{13} + {a}_{14 RR 4} * {e}_{14} + {a}_{23 RR 4} * {e}_{23} + {a}_{24 RR 4} * \\ {e}_{24} + {a}_{33 RR 4} * {e}_{33} + {a}_{34 RR 4} * {e}_{34} + {a}_{43 RR 4} * {e}_{43} + {a}_{44 RR 4} * {e}_{44}\end{matrix}\end{matrix} \qquad [ Math 6 ]$$

And, for receive phase RX2, the following systems:

$$\{ \begin{matrix}\begin{matrix}{V}_{RL 1}^{1} = {a}_{11 RL 1} * {e}_{11} + {a}_{12 RL 1} * {e}_{12} + {a}_{21 RL 1} * {e}_{21} + {a}_{22 RL 1} * \\ {e}_{22} + {a}_{31 RL 1} * {e}_{31} + {a}_{32 RL 1} * {e}_{32} + {a}_{41 RL 1} * {e}_{41} + {a}_{42 RL 1} * {e}_{42}\end{matrix} \\ \begin{matrix}{V}_{R L 2}^{1} = {a}_{11 RL 2} * {e}_{11} + {a}_{12 RL 2} * {e}_{12} + {a}_{21 RL 2} * {e}_{21} + {a}_{22 RL 2} * \\ {e}_{22} + {a}_{31 RL 2} * {e}_{31} + {a}_{32 RL 2} * {e}_{32} + {a}_{41 RL 2} * {e}_{41} + {a}_{42 RL 2} * {e}_{42}\end{matrix} \\ \begin{matrix}{V}_{R L 3}^{1} = {a}_{11 RL 3} * {e}_{11} + {a}_{12 RL 3} * {e}_{12} + {a}_{21 RL 3} * {e}_{21} + {a}_{22 RL 3} * \\ {e}_{22} + {a}_{31 RL 3} * {e}_{31} + {a}_{32 RL 3} * {e}_{32} + {a}_{41 RL 3} * {e}_{41} + {a}_{42 RL 3} * {e}_{42}\end{matrix} \\ \begin{matrix}{V}_{R L 4}^{1} = {a}_{11 RL 4} * {e}_{11} + {a}_{12 RL 4} * {e}_{12} + {a}_{21 RL 4} * {e}_{21} + {a}_{22 RL 4} * \\ {e}_{22} + {a}_{31 RL 4} * {e}_{31} + {a}_{32 RL 4} * {e}_{32} + {a}_{41 RL 4} * {e}_{41} + {a}_{42 RL 4} * {e}_{42}\end{matrix}\end{matrix} \qquad [ Math 5 ]$$

$$\{ \begin{matrix}\begin{matrix}{V}_{RR 1}^{1} = {a}_{13 RR 1} * {e}_{13} + {a}_{14 RR 1} * {e}_{14} + {a}_{23 RR 1} * {e}_{23} + {a}_{24 RR 1} * \\ {e}_{24} + {a}_{33 RR 1} * {e}_{33} + {a}_{34 RR 1} * {e}_{34} + {a}_{43 RR 1} * {e}_{43} + {a}_{44 RR 1} * {e}_{44}\end{matrix} \\ \begin{matrix}{V}_{RR 2}^{1} = {a}_{13 RR 2} * {e}_{13} + {a}_{14 RR 2} * {e}_{14} + {a}_{23 RR 2} * {e}_{23} + {a}_{24 RR 2} * \\ {e}_{24} + {a}_{33 RR 2} * {e}_{33} + {a}_{34 RR 2} * {e}_{34} + {a}_{43 RR 2} * {e}_{43} + {a}_{44 RR 2} * {e}_{44}\end{matrix} \\ \begin{matrix}{V}_{RR 3}^{1} = {a}_{13 RR 3} * {e}_{13} + {a}_{14 RR 3} * {e}_{14} + {a}_{23 RR 3} * {e}_{23} + {a}_{24 RR 3} * \\ {e}_{24} + {a}_{33 RR 3} * {e}_{33} + {a}_{34 RR 3} * {e}_{34} + {a}_{43 RR 3} * {e}_{43} + {a}_{44 RR 3} * {e}_{44}\end{matrix} \\ \begin{matrix}{V}_{RR 4}^{1} = {a}_{13 RR 4} * {e}_{13} + {a}_{14 RR 4} * {e}_{14} + {a}_{23 RR 4} * {e}_{23} + {a}_{24 RR 4} * \\ {e}_{24} + {a}_{33 RR 4} * {e}_{33} + {a}_{34 RR 4} * {e}_{34} + {a}_{43 RR 4} * {e}_{43} + {a}_{44 RR 4} * {e}_{44}\end{matrix}\end{matrix} \qquad [ Math 6 ]$$

It should be noted that, as discussed in the above-mentioned patent application WO2022/268512, the reversal of the sign of bias voltage V_{bias} and of excitation voltage V_{exc} during the transmit phase has no effect on the transmitted ultrasonic wave. Thus, it would be possible to perform both firings with the same bias and excitation voltages, and to change the sign of the bias voltage of columns C₂ and C₄ only during receive phase RX2. In practice, it may however be difficult to reverse the sign of the bias voltage of a column between the phase of transmission and the phase of reception of a same ultrasonic wave. The sign of the bias voltage of the electrode of columns C₂ and C₄ will thus preferably be modified from as soon as the transmit phase.

Those skilled in the art will be capable of adapting the described method regardless of the number N of rows and columns in the device.

For an array of N×N size, it will be sufficient to perform N/2 successive firings TXN/2, each time changing the sign of the bias voltage applied to at least one of the column electrodes of the array during the respective subsequent receive phase RX1, . . . . RXN/2.

For each of receive phases RX1 and RXN/2, two equation systems of the same type as the systems [Math 5] and [Math 6] described hereabove can be written, with, each time, N equations per system and N*(N/2) terms a_{ijRLi}*eᵢⱼ or a_{ijRRi}*eᵢⱼ per equation.

For each ultrasound firing, a set of 2N equations and N*N unknowns is thus obtained.

With N/2 ultrasonic firings, N*N equations for N*N unknowns are thus obtained, which enables to trace back to the individual contributions eᵢⱼ of all the elementary transducers.

As an example, the signs of the bias voltages respectively applied to the N column electrodes C₁, . . . . CN during the N/2 receive phases RX1, . . . . RXN/2 are encoded by vectors of an orthogonal matrix, for example a Hadamard matrix, for example the first N/2 vectors of a Hadamard matrix of size N. By using N/2 linear combinations of the voltage measurements performed on the row electrodes RLᵢ, RRᵢ of the array, it is possible to trace back to the individual contributions eᵢⱼ of the N*N elementary transducers of the device.

According to a preferred embodiment, at each firing of the ultrasonic wave, the bias voltage applied to each of the column electrodes Cⱼ, with j ranging from 1 to N/2, is equal to the bias voltage applied to the column electrode of one of columns C_{(N/2)+1}, . . . , C_{N}, for example column C_{j+(N/2)}.

In this case, a same transmit circuit, for example a same pulse generator, may be used to simultaneously control the electrodes of two columns.

This advantageously enables to divide by two the number of transmit circuits of the device. Thus, as compared with a configuration of the type described in relation with FIGS. 1 to 4, the number of row receive circuits is doubled (two receive circuits per row of the array instead of a single one in the examples of FIGS. 1 to 4), and the number of column transmit circuits is divided by two (one transmit circuit for two columns of the array).

An advantage of the provided embodiments is that they enable to benefit both from the advantages of fully populated devices in terms of imaging resolution and of row-column addressing devices in terms of electronic complexity.

Further, as compared with the solution described in above-mentioned patent application WO2022/268512, the number of firings of a same ultrasonic wave required for the reconstruction of a fully resolved image is decreased.

It should be noted that the described embodiments are not limited to the example described hereabove, in which each row of ultrasonic transducers in the array is divided into two row portions R_{Li}, R_{Ri}. More generally, those skilled in the art will be capable of adapting the described operation to a device in which each row of the ultrasonic transducer array is divided into n row portions of same dimensions, with n an integer greater than or equal to 2, N a multiple of n, and N/n an integer greater than or equal to 2.

In this case, the number of ultrasonic firings required to reconstruct the individual contributions eᵢⱼ of all the elementary transducers of the array will be decreased to N/n, at the cost of an increase by a factor n in the number of receive circuits (equal to n*N) respectively connected to the electrodes of the n*N row portions of the device. Advantageously, a same transmit circuit may be used to simultaneously control n columns of the array, decreasing to N/n the number of transmit circuits of the device.

Further, those skilled in the art will be capable of adapting the described embodiments to cases where the row portions do not all comprise the same number of ultrasonic transducers.

Further, those skilled in the art will be capable of adapting the described embodiments to cases where the row portions are formed of non-necessarily adjacent transducers. For example, for a given row of ultrasonic transducers, the transducers in the columns of even rank may be connected to a first row electrode defining a first row portion, and the transducers of the columns of odd rank may be connected to a second row electrode distinct from the first electrode (that is, not connected to the first electrode), defining a second row portion.

Various embodiments and variants have been described. Those skilled in the art will understand that certain features of these various embodiments and variants could be combined, and other variants will be apparent to those skilled in the art. In particular, the described embodiments are not limited to the specific example of the elementary transducer array described in relation with FIG. 8.

Further, the described embodiments are not limited to devices based on CMUT-type ultrasonic transducers, but more generally apply to any type of transducer exhibiting symmetrical behavior, particularly in receive mode, for example PMUT (Piezoelectric Micromachined Ultrasonic Transducers) transducers. By symmetrical behavior in receive mode, there is here meant that the sign of the AC voltage generated by the transducer during a phase of reception of an ultrasonic wave is reversed when the sign of the DC bias voltage applied to the transducer during this same receive phase is reversed.

Further, although in the examples described hereabove, the DC bias voltages V_{bias} and −V_{bias} applied to the different column electrodes Cⱼ of the device all have the same amplitude in absolute value, the described embodiments are not limited to this specific case. As a variant, the amplitude levels of the DC bias voltages applied to the different column electrodes Cⱼ, in transmit and/or receive mode, may be differentiated. Similarly, the AC excitation voltages V_{exc} applied to the column electrodes Cⱼ in transmit mode may be differentiated by column.

Further, the described embodiments are not limited to the examples detailed hereabove, in which the reversal of the sign of the contribution eᵢⱼ of the elementary transducers of a column is reversed by reversing the sign of the DC bias voltage V_{bias} applied to this column. More generally, any other means enabling to reverse the sign of the contribution eᵢⱼ of one or more elementary transducers between two successive firings of the same ultrasonic wave may be provided. As an example, each elementary transducer at position (i,j) in the array is associated with a system of switches, for example, electromechanical switches, enabling to reverse the connection direction of the transducer electrodes between the row electrodes Rᵢ and the column electrodes Cⱼ of the array. In other words, in a first configuration of the switch system, the transducer has a first electrode connected to electrode Rᵢ and a second electrode connected to electrode Cⱼ, and, in a second configuration of the switch system, the transducer has its first electrode connected to electrode Cⱼ its second electrode connected to electrode Rᵢ. Thus, by changing, between two successive firings of the same ultrasonic wave, the configuration of the switch system associated with an elementary transducer of position (i,j), the sign of the contribution eᵢⱼ of this transducer is reversed. This enables to increase the number of discriminating equations in the system and thus to trace back to the individual contributions eᵢⱼ of all the elementary transducers in the array. In this embodiment, the switch systems associated with the individual transducers can be controlled individually, transducer by transducer, or simultaneously, in columns. It should be noted that this embodiment is compatible with any type of ultrasonic transducers, including transducers which do not exhibit a symmetrical voltage behavior. In particular, this embodiment is not limited to CMUT and PMUT transducers.

Further, although examples of implementation based on voltage measurements on the electrodes of the device have been described hereabove, the described embodiments are not limited to this specific case. As a variant, the described embodiments may be adapted to tracing back to the individual contributions of elementary transducers based on measurements of another variable electrical quantity, for example a current, charges, or an impedance, on the electrodes of the device.

Further, although the above-detailed examples of the provided acquisition method have been given for square transducer arrays of N rows and N columns, the described embodiments are not limited to this specific case. On reading of the present disclosure, those skilled in the art will be capable of adapting the provided method to array devices having a number of rows different from the number of columns.

Further, in the above-described embodiments, at each firing of the ultrasonic wave, the wave is generated by the same array device as that used for the reception of the return wave. The described embodiments are however not limited to this specific case. As a variant, the transducer array is used only as a receive device, to receive the ultrasonic wave returned by the body to be analyzed, and a separate transmit device (not detailed) is used to transmit the ultrasonic wave toward the body to be analyzed. The transmit device and the receive device are then synchronized to implement the alternated N/n transmit phases TX and N/n receive phases RX.

Further, the practical implementation of the described embodiments and variants is within the abilities of those skilled in the art based on the functional indications given hereabove. In particular, the forming of the electronic control circuits of the transmit and receive devices to implement the provided method has not been detailed, the forming of these circuits being within the abilities of those skilled in the art on reading of the present disclosure. Further, the forming of the electronic processing devices enabling to trace back to the individual contributions of the elementary transducers from the quantities measured on the electrodes of the device has not been detailed, the implementation of such devices being within the abilities of those skilled in the art based on the teachings of the present disclosure.

Further, it should be noted that in the above-described examples, the terms “row” and “column” are arbitrary and can of course be inverted.

1. Method of acquisition of an image of a body by means a row-column addressing array ultrasonic imaging device, the device comprising an array of M rows and N columns of elementary ultrasonic transducers, each row being divided into n row portions, each row portion comprising a plurality of elementary ultrasonic transducers, with n an integer greater than or equal to two, wherein:
the elementary transducers of a same row portion are connected to a same row portion electrode;
the elementary transducers of distinct row portions are connected to distinct row portion electrodes; and
the elementary transducers of a same column are connected to a same column electrode, the method comprising:
performing p successive firings of a same ultrasonic wave toward the body, where p is an integer designating the number of elementary ultrasonic transducers of the largest row portion;
after each firing, implementing a phase of reception, by means of the device, of a return ultrasonic wave reflected by the body,
wherein, during each of the receive phases, a variable electrical quantity representative of the received wave is read from each row portion electrode of the device,
and wherein, between any two receive phases among the p receive phases, the sign of the individual contribution of at least one elementary ultrasonic transducer of the array is changed,
the method further comprising a step of calculating, by means of an electronic processing device, by linear combinations of the variable electrical quantities read from the row portion electrodes of the device during the p receive phases, an individual contribution of each of the elementary transducers of the array.

2. Method according to claim 1, wherein the row portions all comprise the same integer number N/n of elementary ultrasonic transducers.

3. Method according to claim 1, wherein the number M of rows of the array is equal to the number N of columns of the array.

4. Method according to claim 1, wherein the calculation of the individual contributions of the elementary transducers of the array comprises a multiplication of the variable electrical quantities read from the row portion electrodes of the device during the p receive phases by coefficients of an array, determined in advance during a characterization or simulation phase and stored in a memory of the electronic processing device.

5. Method according to claim 1, wherein, during each of the p receive phases, each column electrode of the array is maintained at a DC bias voltage, and wherein, between any two receive phases among the p receive phases, the sign of the DC bias voltage applied to at least one of the column electrodes of the device is changed.

6. Method according to claim 5, wherein the signs of the bias voltages respectively applied to the column electrodes of the device during the p receive phases are encoded by vectors of an orthogonal matrix, for example a Hadamard matrix.

7. Method according to claim 5, wherein the p successive firings are performed by means of the row-column addressing array ultrasonic imaging device, and wherein, during each of the p firings, each column electrode of the array is maintained at a DC bias voltage, and an AC excitation voltage superimposed on the DC bias voltage is applied to said column electrode, and wherein, for each firing, the signs of the DC bias voltages respectively applied to the column electrodes of the array are the same as the signs of the DC bias voltages respectively applied to the column electrodes during the subsequent receive phase.

8. Method according to claim 7, wherein, for each firing, a same transmit circuit simultaneously applies a same DC bias voltage and a same AC excitation voltage to the column electrodes of n columns of the array.

9. Method according to claim 1, wherein the ultrasonic transducers are CMUT or PMUT transducers.

10. Method according to claim 1, wherein n is equal to two.

11. Method according to claim 1, wherein the variable electrical quantity read from each row portion electrode of the device is a voltage.

12. Row-column addressing array ultrasonic imaging device, the device comprising an array of M rows and N columns of elementary ultrasonic transducers, each row being divided into n row portions, each row portion comprising a plurality of elementary ultrasonic transducers, with n an integer greater than or equal to two, wherein:
the elementary transducers of a same row portion are connected to a same row portion electrode;
the elementary transducers of distinct row portions are connected to distinct row portion electrodes; and
the elementary transducers of a same column are connected to a same column electrode,
the device further comprising a control circuit configured to implement a method according to claim 1.

13. Device according to claim 12, comprising M*n receive circuits respectively connected to the M*n row portion electrodes of the array of elementary ultrasonic transducers.

14. Device according to claim 12 or 13, comprising an integer s smaller than N of transmit circuits, where a same transmit circuit may be simultaneously connected to column electrodes of elementary transducers belonging to distinct row portions of a same row of the array.

15. Device according to claim 14, wherein the row portions all comprise the same integer number N/n of elementary ultrasonic transducers, and wherein the number s of transmit circuits is equal to N/n.
